(19) United States
(12) Patent Application Publication
(10) Pub. No.: US 2026/0241162 A1
(43) Pub. Date: Aug. 20, 2026

(54) EXTERNAL PACING GROUNDING INTRODUCER

(71) Applicant: BOSTON SCIENTIFIC SCIMED, INC., MAPLE GROVE, MN (US)

(72) Inventors: Matthew Hart, Maple Grove, MN (US); Andrew John Mitterholzer, Eden Prairie, MN (US); Luis Gabriel Lopez-de-Victoria, Maple Grove, MN (US)

(73) Assignee: BOSTON SCIENTIFIC SCIMED, INC., MAPLE GROVE, MN (US)

(21) Appl. No.: 19/543,324

(22) Filed: Feb. 18, 2026

Related U.S. Application Data
(60) Provisional application No. 63/759,702, filed on Feb. 18, 2025.

(51) Int. Cl. A61N 1/05; A61M 39/10; A61M 25/00; A61M 25/06

(52) U.S. Cl. CPC A61N 1/056; A61M 39/10; A61M 25/0045; A61M 25/005; A61M 2025/0681; A61M 2039/1022

(57) ABSTRACT

An introducer for use during a transcatheter procedure includes a tubular sheath having a proximal end and a coil extending distally from the proximal end of the tubular sheath. The coil is encapsulated in a polymer coating having first and second cutout portions that expose the coil to define distal and proximal contact portions. A hub at the proximal end of the tubular sheath circumferentially surrounds the proximal region of the coil and includes the proximal contact portion. The introducer enables simplified electrical connections during transcatheter procedures by providing a secure electrical pathway between patient tissue and an external pulse generator through the exposed coil portions.

### CROSS REFERENCE TO RELATED APPLICATIONS

This application claims the benefit of priority of U.S. Provisional Application No. 63/759,702 filed February 18, 2025, the entire disclosure of which is hereby incorporated by reference.

### TECHNICAL FIELD

The present disclosure generally relates to establishing electrical connections during transcatheter procedures. Particularly, but not exclusively, the present disclosure relates to strengthening electrical connections of a pacing system used during transcatheter procedures via a simplified circuit configuration.

### BACKGROUND

Transcatheter procedures often require establishing temporary electrical connections to provide cardiac pacing. Traditional approaches for establishing these connections typically require multiple vascular access sites and additional devices or users, which can increase procedural complexity and patient discomfort.

Current methods of establishing electrical connections during procedures like transcatheter aortic valve replacement (TAVR) can result in inconsistent electrical contact and potential signal loss. There remains a need for reliable solutions that can maintain consistent electrical contact with patient tissue while reducing procedural complexity and the number of vascular access sites required.

### BRIEF SUMMARY

This overview is intended to provide an introduction to the subject matter of the present patent application. It is not intended to provide an exclusive or exhaustive explanation. The detailed description is included to provide further information about the present patent application.

In an example, an introducer for use during a transcatheter procedure includes a tubular sheath having a proximal end, a coil having a distal region and a proximal region extending distally from the proximal end of the tubular sheath, and a polymer coating at least partially encapsulating the coil. The polymer coating includes a first cut-out portion in the distal region of the coil exposing the coil to define a distal contact portion, and a second cut-out portion in the proximal region of the coil exposing the coil to define a proximal contact portion. The introducer has a hub at the proximal end of the tubular sheath, wherein the hub surrounds the proximal region of the coil and includes the proximal contact portion.

Alternatively or additionally to any of the examples above, the distal contact portion is positioned and configured to be in direct contact with a patient tissue when inserted through a vascular access site.

Alternatively or additionally to any of the examples above, the proximal contact portion is configured to connect to a negative lead of an external pulse generator via an electric connector.

Alternatively or additionally to any of the examples above, the hub includes a metal connector configured to be in contact with the exposed coil at the proximal contact portion.

Alternatively or additionally to any of the examples above, the metal connector is a conductive film applied circumferentially to the hub.

Alternatively or additionally to any of the examples above, the metal connector is integrated into the hub during a hub molding process.

Alternatively or additionally to any of the examples above, the metal connector is configured to connect to a negative lead of an external pulse generator via an electric connector.

Alternatively or additionally to any of the examples above, the axial length of the proximal contact portion is at least 1 millimeter.

Alternatively or additionally to any of the examples above, the axial length of the distal contact portion is at least 1 millimeter.

In an example, a pacing introducer for use during a transcatheter procedure includes a tubular sheath having a proximal end region, a metal coil disposed circumferentially around the tubular sheath at the proximal end region, the metal coil having a distal region and a proximal region, and a polymer coating at least partially encapsulating the metal coil. The polymer coating includes a first cut-out portion in the distal region of the metal coil exposing the metal coil to define a distal electrical contact portion. The pacing introducer has a hub disposed on the proximal end region of the tubular sheath, wherein the hub surrounds the proximal region of the metal coil and defines a proximal electrical contact portion.

Alternatively or additionally to any of the examples above, the proximal electrical contact portion includes a second cut-out portion of the polymer coating exposing the metal coil.

Alternatively or additionally to any of the examples above, the distal contact portion is configured to be in direct contact with a patient tissue when the tubular sheath is inserted through a vascular access site with just the hub positioned outside the patient.

Alternatively or additionally to any of the examples above, the proximal electrical contact portion includes a metal connector configured to be in contact with the metal coil at the proximal contact portion.

Alternatively or additionally to any of the examples above, the axial length of the proximal or distal contact portion is at least 5 millimeters.

Alternatively or additionally to any of the examples above, the proximal electrical contact portion includes a conductive film configured to be in contact with an exposed portion of the metal coil at the proximal contact portion.

In an example, a method of establishing electrical pacing during a transcatheter procedure includes advancing an introducer into a patient's vasculature through a vascular access site. The introducer includes a tubular sheath having a proximal end, a coil having a distal region and a proximal region extending distally from the proximal end of the tubular sheath, and a polymer coating at least partially encapsulating the coil. The polymer coating includes a first cut-out portion in the distal region of the coil exposing the coil to define a distal contact portion, and a second cut-out portion in the proximal region of the coil exposing the coil to define a proximal contact portion. The introducer has a hub at the proximal end of the tubular sheath, wherein the hub surrounds the proximal region of the coil and includes the proximal contact portion. The method includes advancing the introducer by placing the distal contact portion of the introducer in contact with a tissue of the patient, connecting a positive lead from a positive terminal of an external pulse generator to a guidewire advanced into the patient's vasculature, connecting a negative lead from a negative terminal of the external pulse generator to the proximal contact portion of the introducer, and delivering electrical stimuli through an electrical pathway including the positive and negative leads during the transcatheter procedure.

Alternatively or additionally to any of the examples above, the introducer is advanced to a length such that the hub remains proximal to the vascular access site.

Alternatively or additionally to any of the examples above, the negative lead is connected to the proximal contact portion of the introducer via an electrical connector.

Alternatively or additionally to any of the examples above, the hub of the introducer includes a metal connector configured to be in contact with the coil exposed at the proximal contact portion.

Alternatively or additionally to any of the examples above, the negative lead is connected to the metal connector via an electrical connector.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a schematic diagram illustrating electrical connections of a pacing system during a transcatheter procedure utilizing an introducer.

FIG. 2 is a schematic side view of the introducer in accordance with an embodiment of the subject matter disclosed herein.

FIG. 3A shows a cross-sectional view of a proximal coil region taken through line 3-3 in FIG. 2

FIG. 3B shows a cross-sectional view of the proximal coil region in accordance with another embodiment of the subject matter disclosed herein.

FIG. 3C is a perspective view of the proximal coil region of the introducer from FIG. 2 in accordance with an embodiment of the subject matter disclosed herein.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Direct wire pacing systems may be used to provide temporary pacing to the heart. During a transcatheter procedure, such as transcatheter aortic valve replacement (TAVR), an introducer may provide a safer access to a patient's vasculature and facilitate the placement of a delivery catheter.

A conventional pacing system consists of an external pulse generator (herein referred to as “EPG”), a positive lead connected to a guidewire, and a negative lead connected to a patient. Electrical stimulus begins at the positive terminal of the EPG and flows through the positive lead and the guidewire at an access site to a vasculature of a patient. In a TAVR procedure, the targeted vasculature includes the aortic valve annulus, the aortic root, ascending aorta, and the aortic arch.

Once the stimulus is delivered to the target treatment location, the stimulus then returns to the negative terminal of the EPG through the negative lead. In conventional approaches for establishing a negative terminal connection, a negative lead extending from an external pulse generator is often connected to the patient's abdominal tissue using a needle or alligator clip or by using a secondary tissue-contacting guidewire from a secondary access site. These configurations may be physically insecure and/or require multiple access points and/or users, increasing procedural complexity and potential complications. By including the introducer in establishing the negative terminal connection of the pacing system, the disclosed pacing system provides a more secure electrical pathway.

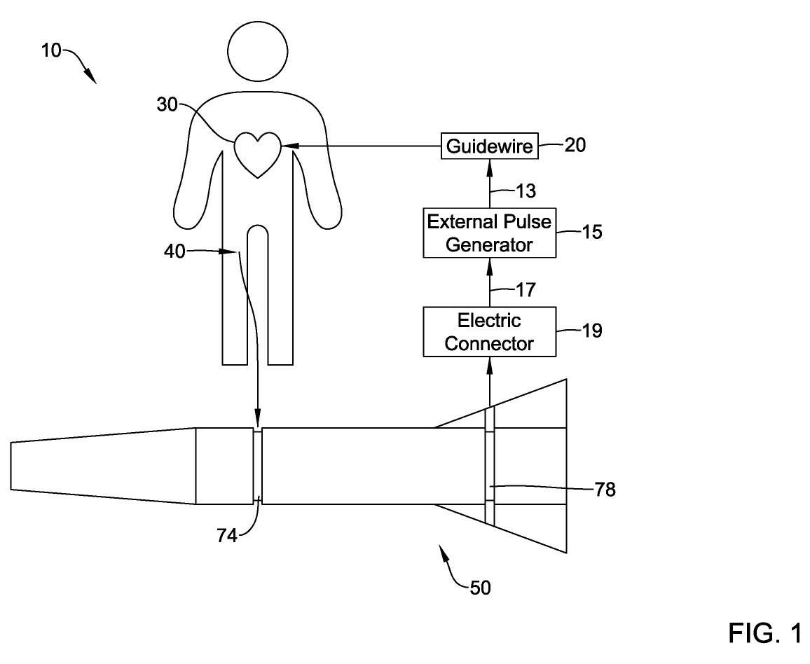

FIG. 1 shows an illustrative pacing system 10. Stimulation begins at a positive terminal of the EPG 15, through a positive lead 13 to a guidewire 20 and to a patient’s vasculature 30. Once the stimulus is delivered to the patient’s vasculature 30, the stimulation is returned to a negative terminal of the EPG 15 via a distal contact portion 74 on an introducer 50 inserted into a vascular access site 40, with a proximal contact portion 78 on the introducer 50 connected to the negative lead 17 via an electric connector 19. The distal contact portion 74 and the proximal contact portion 78 may provide electrical connections. Possible electric connectors 19 may include alligator clips, snap connectors, banana plugs, screw-type connectors, pin and socket connectors, spring loaded clamps, or other connectors made of conductive material.

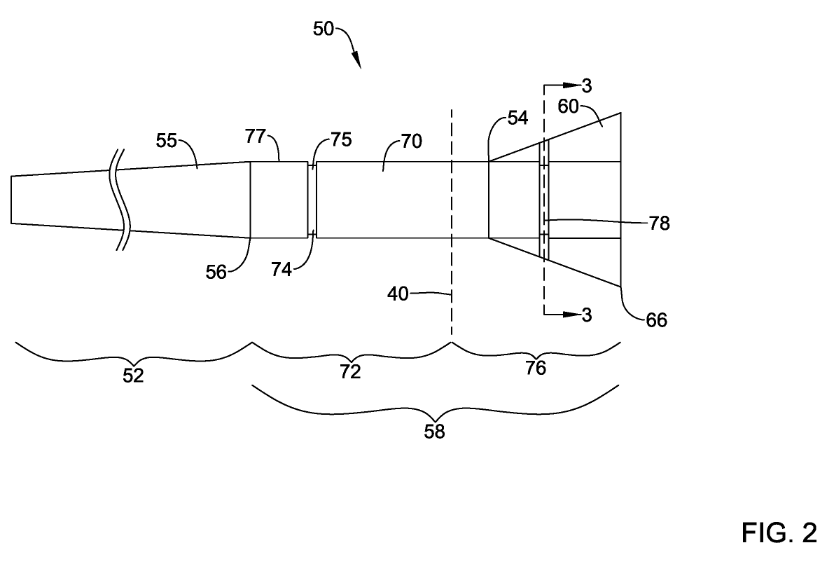

FIG. 2 shows a side view of the introducer 50 comprising of a tubular sheath 55 and a hub 60. Extending from a proximal end 66 of the hub 60 to a proximal end 56 of a distal region 52 of the tubular sheath 55 is a coil 75, overlaying a proximal region 58 of the tubular sheath 55. The hub 60 extends from a distal end 54 of the tubular sheath 55, circumferentially surrounding a proximal region 76 of the coil 75 (herein referred to as the “proximal coil region” 76). The coil 75 may be made of metal or another electrically conductive material and is configured to provide strain relief to the proximal end region of the tubular sheath 55 and the hub 60. The coil 75 may be disposed circumferentially around the tubular sheath 55.

The coil 75 is encapsulated in a polymer coating 77 to ease insertion and provide atraumatic removal of the introducer 50. The polymer coating 77 includes a first cutout over a distal region 72 of the coil 75 (herein referred to as “distal coil region” 72) and a second cutout over the proximal coil region 76. The cut outs of the polymer coating 77 expose the underlying coil 75, providing a conductive surface, with the first cutout defining a distal contact portion 74 and the second cutout defining a proximal contact portion 78.

The distal contact portion 74 is the electrical connection point to the patient. When the introducer 50 is advanced into the patient vasculature 30 through the vascular access site 40 (illustrated by the dashed line) up to the patient vasculature 30, the exposed coil 75 at the distal contact portion 74 is in direct contact with patient tissue at or distal to the vascular access site 40, while the hub 60 with the exposed coil 75 at the proximal contact portion 78 remains outside the patient’s body, proximal to the vascular access site 40 and accessible for connection to an electric connector 19 on the negative lead 17. To ensure consistent and adequate electrical conductivity with the patient tissue, the axial length of the distal contact portion 74 may range from 1 millimeter to 50 millimeters (0.03937 inches to 1.9685 inches).

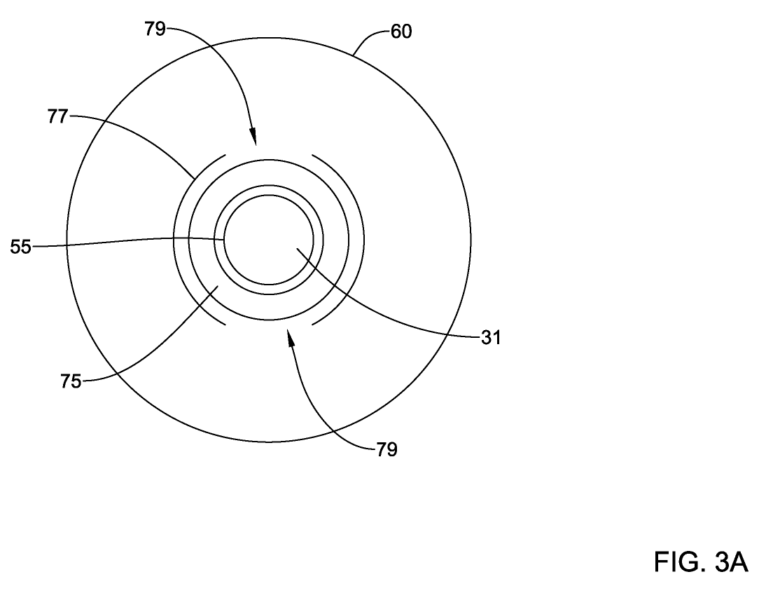

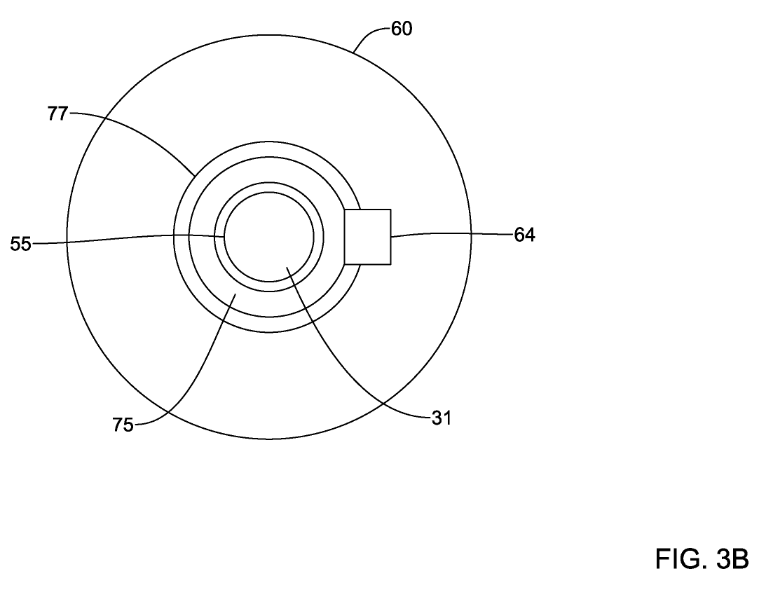

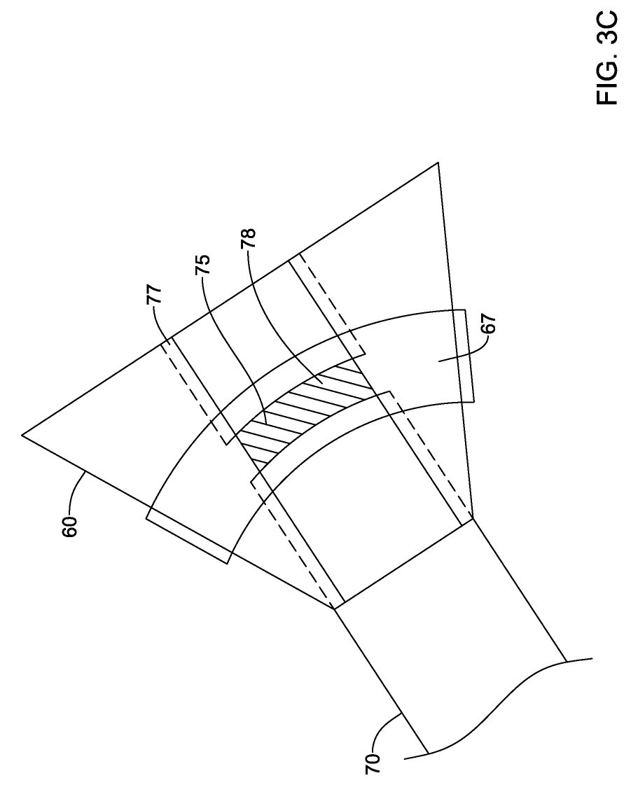

FIG. 3A shows a cross-sectional view of the proximal coil region 76 taken through line 3-3 in FIG. 2, showing the layers of introducer 50 at the proximal contact portion 78. The layers of the proximal coil region 76 as illustrated, starting from its central axis, may include a lumen 31 to receive the delivery catheter, the tubular sheath 55, the coil 75, the polymer coating 77, and the hub 60. As illustrated, the coil 75 is exposed in two regions 79 where the polymer coating 77 has been removed. These regions 79 of exposed coil 75 allow for the electric connector 19 of the negative lead 17 to be coupled directly to the coil 75 to complete the electrical circuit.

In another embodiment, instead of an exposed region of the coil 75, the hub 60 may include a metal connector 64 connected directly to the coil 75 at the proximal contact portion 78. See FIG. 3B. The metal connector 64 may extend radially outward beyond the outer surface of the polymer coating 77. The electrical connector 19 on the negative lead 17 may be clamped directly onto the metal connector 64 to provide an electrical connection to the negative lead 17 in order to provide conductivity through the hub 60 from the proximal contact portion 78 to the negative lead 17 of the EPG 15. As shown in FIG. 3B, to provide the necessary electrical pathway, the metal connector 64 is in electrical contact with the coil at the proximal contact portion 78.

In a further embodiment, and as illustrated in FIG. 3C, a conductive film 67 may be applied circumferentially around the hub 60 such that it contacts the exposed coil 75 at the proximal contact portion 78. The conductive film 67 may be manufactured of material that has electrical conductivity, such as a metal or a metal alloy. Examples of appropriate, but non-exclusive, materials for manufacture of the conductive film 67 include, but are not limited to, stainless steel, titanium, tantalum, tungsten, gold, platinum, palladium, and combinations thereof; and the metal alloy is selected from the group consisting of nickel-titanium alloys, nickel-chromium alloys, nickel-chromium-iron alloys, cobalt-chromium-nickel alloys, nickel-chromium-molybdenum alloys, nickel-molybdenum alloys, nickel-copper alloys, cobalt-chromium alloys, cobalt-chromium-molybdenum alloys, platinum-enriched stainless steel, and combinations and alloys thereof. Other considerations in the selection of the material may include, but are not limited to, strength, flexibility, corrosion resistance, and biocompatibility. Additionally, the manufacturing process may incorporate additional conductive elements, such as nanoparticles, to achieve the necessary electrical properties.

The conductive film 67 may be manufactured and applied to the hub 60 separately or together during the manufacturing process. In other embodiments the conductive film 67 may be applied separately from the manufacturing process as a secondary operation prior to preparation and use of the pacing system. When the conductive film 67 is applied as a secondary operation, potential, but not exclusive, advantages include allowance to use different film materials or specifications may be used without modifying the hub molding process. The conductive film 67 may be inspected separately before application to ensure proper conductivity and quality of the electrical contact surface, and the conductive film 67 may be replaced if damaged without affecting the hub 60 structure.

In other embodiments, the conductive film 67 may be integrated into the hub 60 during the hub molding process. Potential advantages include, but are not limited to, streamlining production by eliminating secondary operations, providing a more secure bond between the hub 60 and the conductive film 67 and potentially reducing the risk of separation during use, and by eliminating the need for potentially non-conductive securing mechanisms to ensure consistent electrical contact with the exposed coil at the proximal contact portion 78.

The conductive film 67 may be secured to the hub 60 in a manner to ensure, among other factors, that the conductive film stays in place, is tight, and does not slip or shift during operation to ensure electrical conductivity. Various methods and mechanisms may be used to secure the metal connector 64 and/or the conductive film 67 to the hub 60. These methods and mechanisms may include, but are not limited to, adhesive bonding, thermal bonding, lamination, welding, frictional fitting, crimping, soldering, insert molding, overmolding, insert molding, or utilization of a mechanical means of fastening such as utilizing separate wire or band, clips, clamps, threaded connectors, rivets, pins, or integrated mechanical interlocking features on the polymer coating 77. Any manner of fastening should be done so in a manner to not interfere with the electrical contact between the exposed coil at the proximal contact portion 78 and the conductive film 67 or metal connector 64. For example, the conductive film 67 or metal connector 64 may be adhesively bonded to the hub 60 at the areas of overlap between the hub 60, the conductive film 67 or metal connector 64, and the polymer coating 77.

The axial length of the conductive film 67 may be selected to ensure electrical contact with the exposed coil 75 at the proximal contact portion 78, ensure complete contact with the electrical electric connector 19 and/or to accommodate space needed to secure the conductive film 67 to the hub 60. The axial length may range from 1 millimeter to 50 millimeters (0.03937 inches to 1.9685 inches).

To operate the pacing system 10 and establish electrical pacing for the procedure, the components of the pacing system, including the EPG 15, the positive lead 13, the guidewire 20, the introducer 50, and the negative lead 17, are mechanically connected.

The introducer 50 is first advanced into the patient vasculature 30 through the vascular access site 40. The introducer 50 must be sufficiently advanced into the patient vasculature 30 such that the distal contact portion 74 is at or distal to the vascular access site 40, ensuring sufficient contact between the exposed coil at the distal contact portion 74 and patient tissue.

Once the introducer 50 is advanced into the patient vasculature 30, the positive lead 13 and negative lead 17 may be connected to the rest of the pacing system 10. The positive lead 13, extending from the positive terminal of the EPG 15, is connected to the guidewire 20. In some embodiments, the guidewire 20 may already be advanced into the patient vasculature 30 prior to connection of the guidewire 20 to the EPG 15. In other embodiments, the positive lead 13 may be connected to the guidewire 20 prior to advancing the guidewire 20 into the patient vasculature 30.

The negative lead, extending from the negative terminal of the EPG 15, is connected to the coil 75 within the introducer 50, either directly via a region of exposed coil 75, or indirectly via a metal connector 64 or conductive film 67 that is in electrical contact with the coil at the proximal contact portion 78 via the electric connector 19.

The connection of the positive lead 13 and the negative lead 17 to the pacing system 10 may be done in either order. In other embodiments, the positive lead 13 may be connected to the EPG 15 and the guidewire 20 prior to advancement of the introducer 50 into the vascular access site 40. Once the components of the pacing system 10 have been mechanically connected, the electric pathway is established and the pacing system 10 is ready to deliver electrical stimuli from the EPG 15.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An introducer for use during a transcatheter procedure, comprising:
a tubular sheath having a proximal end;
a coil, having a distal region and a proximal region, extending distally from the proximal end of the tubular sheath;
a polymer coating at least partially encapsulating the coil;
wherein the polymer coating includes:
a first cut-out portion in the distal region of the coil, exposing the coil to define a distal contact portion, and
a second cut-out portion in the proximal region of the coil, exposing the coil to define a proximal contact portion; and
a hub at the proximal end of the tubular sheath, wherein the hub surrounds the proximal region of the coil and includes the proximal contact portion.

2. The introducer of claim 1, wherein the distal contact portion is positioned and configured to be in direct contact with a patient tissue when inserted through a vascular access site.

3. The introducer of claim 1, wherein the proximal contact portion is configured to connect to a negative lead of an external pulse generator via an electric connector.

4. The introducer of claim 1, wherein the hub further comprises a metal connector configured to be in contact with the exposed coil at the proximal contact portion.

5. The introducer of claim 4, wherein the metal connector is a conductive film applied circumferentially to the hub.

6. The introducer of claim 4, wherein the metal connector is integrated into the hub during a hub molding process.

7. The introducer of claim 4, wherein the metal connector is configured to connect to a negative lead of an external pulse generator via an electric connector.

8. The introducer of claim 1, wherein the axial length of the proximal contact portion is at least 1 millimeter.

9. The introducer of claim 1, wherein the axial length of the distal contact portion is at least 1 millimeter.

10. A pacing introducer for use during a transcatheter procedure, comprising:
a tubular sheath having a proximal end region;
a metal coil disposed circumferentially around the tubular sheath at the proximal end region, the metal coil having a distal region and a proximal region;
a polymer coating at least partially encapsulating the metal coil, the polymer coating including a first cut-out portion in the distal region of the metal coil, exposing the metal coil to define a distal electrical contact portion; and
a hub disposed on the proximal end region of the tubular sheath, wherein the hub surrounds the proximal region of the metal coil and defines a proximal electrical contact portion.

11. The pacing introducer of claim 10, wherein the proximal electrical contact portion includes a second cut-out portion of the polymer coating, exposing the metal coil.

12. The pacing system of claim 10, wherein the distal contact portion is configured to be in direct contact with a patient tissue when the tubular sheath is inserted through a vascular access site with just the hub positioned outside the patient.

13. The pacing system of claim 10, wherein the proximal electrical contact portion comprises a metal connector configured to be in contact with the metal coil at the proximal contact portion.

14. The pacing system of claim 10, wherein the axial length of the proximal or distal contact portion is at least 1 millimeter.

15. The pacing system of claim 10, wherein the proximal electrical contact portion includes a conductive film configured to be in contact with an exposed portion of the metal coil at the proximal contact portion.

16. A method of establishing electrical pacing during a transcatheter procedure, comprising:
advancing an introducer into a patient's vasculature through a vascular access site, wherein the introducer comprises:
a tubular sheath having a proximal end;
a coil, having a distal region and a proximal region, the coil extending distally from the proximal end of the tubular sheath;
a polymer coating at least partially encapsulating the coil;
wherein the polymer coating includes:
a first cut-out portion in the distal region of the coil, exposing the coil to define a distal contact portion, and
a second cut-out portion in the proximal region of the coil, exposing the coil to define a proximal contact portion; and
a hub at the proximal end of the tubular sheath, wherein the hub surrounds the proximal region of the coil and includes the proximal contact portion, wherein advancing the introducer includes placing the distal contact portion of the introducer in contact with a tissue of the patient;
connecting a positive lead from a positive terminal of an external pulse generator to a guidewire advanced into the patient’s vasculature;
connecting a negative lead from a negative terminal of the external pulse generator to the proximal contact portion of the introducer; and
delivering electrical stimuli through an electrical pathway including the positive and negative leads during the transcatheter procedure.

17. The method of claim 16, wherein the introducer is advanced to a length such that the hub remains proximal to the vascular access site.

18. The method of claim 16, wherein the negative lead is connected to the proximal contact portion of the introducer via an electrical connector.

19. The method of claim 16, wherein the hub of the introducer further comprises a metal connector configured to be in contact with the coil exposed at the proximal contact portion.

20. The method of claim 19, wherein the negative lead is connected to the metal connector via an electrical connector.
